(19) United States
(12) Patent Application Publication
(10) Pub. No.: US 2018/0197625 A1
(43) Pub. Date: Jul. 12, 2018

(54) System For Measuring and Tracking Health Behaviors To Implement Health Actions

(71) Applicant: David Lobach, Durham, NC (US)

(72) Inventor: David Lobach, Durham, NC (US)

(21) Appl. No.: 15/868,208

(22) Filed: Jan. 11, 2018

Related U.S. Application Data
(60) Provisional application No. 62/445,056, filed on Jan. 11, 2017.

(51) Int. Cl. G16H 10/60; G06Q 40/08; G16H 10/20; G16H 20/60

(52) U.S. Cl. CPC G16H 10/60; G16H 20/60; G16H 10/20; G06Q 40/08

(57) ABSTRACT

A health index system comprises a processor which executes a behavior unit, an index unit, and an output unit. The behavior unit is configured to receive an accumulated health data for a patient from a behavior database of a health information system and filter the accumulated health data into a patient modifiable data by including in the patient modifiable data only data related to clinical and non-clinical patient modifiable behaviors. The index unit is configured to receive the patient modifiable data from the behavior unit and calculate a health index for the patient from the patient modifiable data. The output unit is configured to receive the health index from the index unit and transmit the health index to an index database of the health information system.

### CROSS-REFERENCE TO RELATED APPLICATION

This application claims priority under 35 U.S.C. § 119 to U.S. Provisional Patent Application No. 62/445,056 filed Jan. 11, 2017.

### FIELD OF THE INVENTION

The present invention relates to a healthcare resource system, and more particularly, to a system for measuring and tracking health behaviors to implement health actions.

### BACKGROUND

Many known measures exist for monitoring and tracking health behaviors. An individual or patient has information such as age, weight, tobacco use, prescription medications filled, clinical services used, and studies performed among myriad other information stored and tracked within various health records. Increasingly, patients can also use technology to track measurements related to personal health behaviors such as steps taken, calories burned, blood glucose levels, and calories consumed to make health behavior decisions.

Modern sources of health data are as numerous as they are disparate; an assessment of a patient's individual health requires gathering and separately considering information in many different formats. Further, a patient's health is often assessed as a measure of factors characterizing the presence or absence of illness existing at one point in time. A patient, for example, is considered less healthy if he or she is currently suffering from or prone to a disease beyond his or her control. Recent advances in the healthcare field, however, suggest that health behaviors modifiable by the patient, not just the presence or absence of disease, are a major factor in the cost and long-term quality of healthcare. No system is currently capable of quantifying and tracking a single, standardized metric measuring a patient's engagement in patient-modifiable health behaviors or using such a metric to implement health actions.

### SUMMARY

A health index system comprises a processor which executes a behavior unit, an index unit, and an output unit. The behavior unit is configured to receive an accumulated health data for a patient from a behavior database of a health information system and filter the accumulated health data into a patient modifiable data by including in the patient modifiable data only data related to clinical and non-clinical patient modifiable behaviors. The index unit is configured to receive the patient modifiable data from the behavior unit and calculate a health index for the patient from the patient modifiable data. The output unit is configured to receive the health index from the index unit and transmit the health index to an index database of the health information system.

### BRIEF DESCRIPTION OF THE DRAWINGS

The invention will now be described by way of example with reference to the accompanying figures, of which:

FIG. 1 is a schematic diagram of a healthcare resource system according to the invention;

FIG. 2 is a schematic diagram of a patient population, a provider, and an insurer of the healthcare resource system;

FIG. 3 is a flow diagram of a process of a behavior unit of a health index system of the healthcare resource system;

FIG. 4 is a schematic diagram of a subcomponent calculation algorithm of an index unit of the health index system;

FIG. 5 is a schematic diagram of a health behavior measure of the subcomponent calculation algorithm;

FIG. 6 is a flow diagram of a process of the subcomponent calculation algorithm;

FIG. 7 is a flow diagram of a process of an index weighting algorithm of the index unit;

FIG. 8 is a flow diagram of a process of a data quality unit of the health index system;

FIG. 9 is a flow diagram of a process of a patient activation unit of the health index system;

FIG. 10 is a first schematic diagram of a patient interface of the healthcare resource system;

FIG. 11 is a second schematic diagram of the patient interface;

FIG. 12 is a third schematic diagram of the patient interface;

FIG. 13 is a fourth schematic diagram of the patient interface;

FIG. 14 is a fifth schematic diagram of the patient interface;

FIG. 15 is a first schematic diagram of a provider interface of the healthcare resource system;

FIG. 16 is a second schematic diagram of the provider interface;

FIG. 17 is a flow diagram of a process of executing a score alert of the healthcare resource system;

FIG. 18 is a flow diagram of a process of executing an appointment alert of the healthcare resource system; and

FIG. 19 is a flow diagram of a process of executing a plurality of insurer actions of the healthcare resource system.

### DETAILED DESCRIPTION OF THE EMBODIMENT(S)

Exemplary embodiments of the present invention will be described hereinafter in detail with reference to the attached drawings, wherein like reference numerals refer to like elements. The present invention may, however, be embodied in many different forms and should not be construed as being limited to the embodiments set forth herein; rather, these embodiments are provided so that the present disclosure will be thorough and complete, and will fully convey the concept of the disclosure to those skilled in the art.

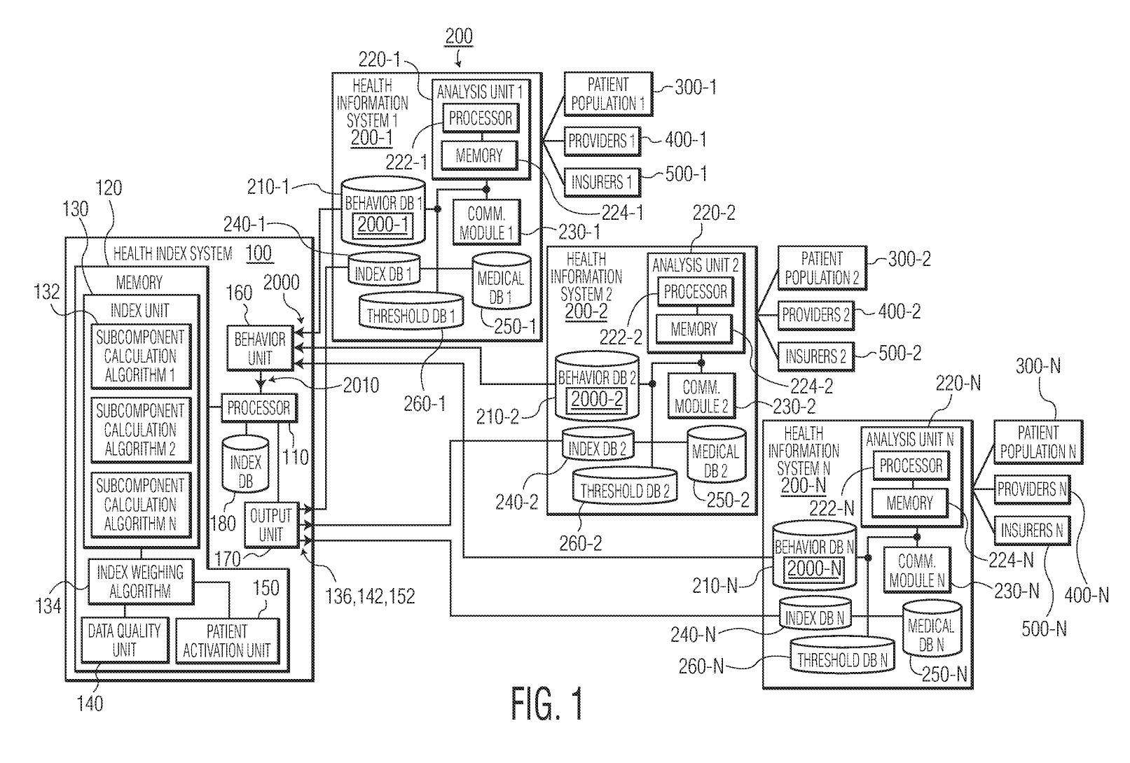

A healthcare resource system 1 according to the invention is shown generally in FIG. 1. The healthcare resource system 1 includes a health index system 100, a plurality of health information resources 200-1 . . . N, a plurality of patient populations 300-1 . . . N, a plurality of providers 400-1 . . . N, and a plurality of insurers 500-1 . . . N.

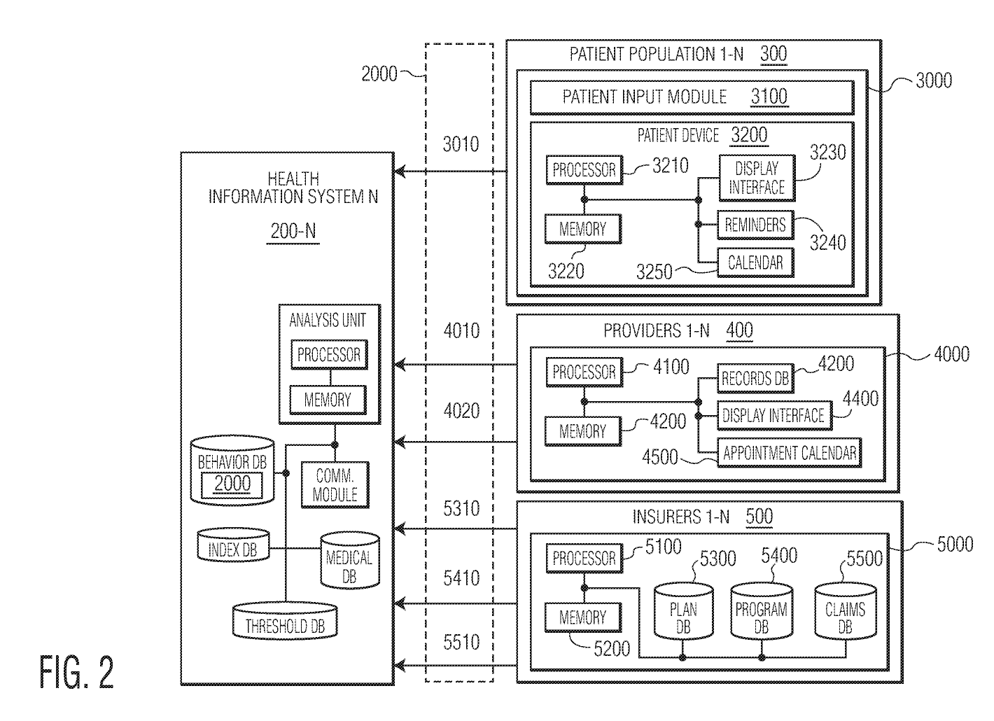

The health index system 100 receives accumulated health data 2000 for each individual patient 3000 from the patient populations 300-1 . . . N, the providers 400-1 . . . N, and the insurers 500-1 . . . N via the health information systems 200, as will now be described with reference to FIGS. 1 and 2.

Each health information system 200-1 . . . N shown in FIG. 1 obtains data from the patient population 300-1 . . . N, providers 400-1 . . . N, and insurers 500-1 . . . N particular to the health information system 200. For example, health information system 200-1 obtains health data from patient population 300-1, providers 400-1, and insurers 500-1, health information system 200-2 obtains health data from patient population 300-2, providers 400-2, and insurers 500-2, and health information system 200-N likewise obtains health data from patient population 300-N, providers 400-N, and insurers 500-N. For simplicity the plurality of health information resources 200-1 . . . N, the plurality of patient populations 300-1 . . . N, the plurality of providers 400-1 . . . N, and the plurality of insurers 500-1 . . . N may hereinafter be referred to in the singular but it should be understood that in each instance, the description could also apply to a plurality. In an embodiment, each health information system 200 is a healthcare network including numerous providers 400 such as hospitals, primary care physicians, other specialty physicians, pharmacies, and any other known healthcare provider capable of retaining any health data on individual patients 300. Each health information system 200 is related to the patient population 300 using the services of the respective providers 400 and an insurer 500 or plurality of insurers 500 providing insurance to the patient population 300 to use at the providers 400 in the healthcare network. In other embodiments, each health information system 200 may be any entity retaining any quantity of health information on a particular patient population 300, such as a self-insured employer.

As shown in FIG. 2, each patient 3000 within a patient population 300 has a patient input module 3100 and a patient device 3200. The patient input module 3100 receives individual data 3010 of the patient 3000 input by the individual patient 3000. In various embodiments, the patient input module 3100 may include individual data 3010 from a health questionnaire, an electronic device such as a wearable activity tracker, or any other device that enables the patient 3000 to convey his or her individual data 3010.

The patient device 3200, as shown in FIG. 2, is an electronic device of the patient 3000 such as a mobile device, computer, or tablet. The patient device 3200 includes a processor 3210 and a memory 3220 connected to the processor 3210. The memory 3220 is a non-transitory computer readable medium capable of storing program instructions executable by the processor 3210. The patient device 3200 also includes a display interface 3230, a reminder application 3240, and a calendar application 3250 connected to the memory 3220 and the processor 3210. In other embodiments, the patient device 3200 may include additional applications for accumulating health data or for taking action based on health data.

The patient input module 3100 and the patient device 3200 output the individual data 3010 to the health information system 200 under control of the processor 3210. The individual data 3010 may include data on gender, age, weight, body mass index (hereinafter, “BMI”), heart rate, activity level, specific instances of exercise, engagement in unhealthy activities such as drinking or smoking, and any other health-based information an individual could provide. In an embodiment, the patient input module 3100 and the patient display 3200 are embodied in the same device.

Each provider 400 of the plurality of providers 400 corresponding to a health information system 200 has a provider computing system 4000 as shown in FIG. 2. Each provider computing system 4000 has a processor 4100 and a memory 4200 connected to the processor 4100. The memory 4200 is a non-transitory computer readable medium capable of storing program instructions executable by the processor 4100. The provider computing system 4000 also includes a records database 4300, a display interface 4400, and an appointment calendar application 4500 connected to the memory 4200 and processor 4100. In other embodiments, the provider computing system 4000 may include additional applications for accumulating health data or for taking action based on health data.

The records database 4300 stores provider data 4010 organized by each individual patient 3000. The records database 4300, and all databases described herein, is embodied as a non-transitory computer readable medium and may be any type of database known to those with ordinary skill in the art. A provider 400, as described above, may be a hospital, primary care physician, other specialty physician, pharmacist, care manager, social worker, physical therapist, nurse educator, and any other known healthcare provider capable of retaining any health data on individual patients 300. The provider data 4010 may include data for each individual patient 3000 including but not limited to hospital admissions, discharge, transfer, inpatient visits, emergency visits, electronic health records, studies performed, pharmacy records, and any other forms of health data known by the provider 4000. The provider computing system 4000, under control of the processor 4100, outputs the provider data 4010 for each individual patient 3000 to the health information system 200.

Each insurer 500 of the plurality of insurers 5001 . . . N corresponding to a health information system has an insurer computing system 5000 as shown in FIG. 2. Each insurer computing system 5000 has a processor 5100 and a memory 5200 connected to the processor 5100. The memory 5200 is a non-transitory computer readable medium capable of storing program instructions executable by the processor 5100. The insurer computing system 5000 also includes a plan database 5300, a program database 5400, and a claims database 5500 connected to the memory 5200 and the processor 5100. In other embodiments, the insurer computing system 5000 may include additional computing components and/or databases for accumulating health data or for taking action based on health data. Each insurer 500 may be a health insurance corporation, a division of a health insurance corporation, or an insurance branch of a self-insured employer.

The plan database 5300 stores plan data 5310 on insurance plans organized by an individual patient 3000 including scope of coverage, monthly premium, copayments, deductible, and any other information relevant to the insurance plan. The program database 5400 stores program data 5410 on health-based incentive programs of the insurer 500, for example, weight control goals, exercise goals, smoking cessation programs, and any other health-based incentive program implementable by an insurer 500. The program data 5410 is linked to the plan data 5310 such that completion of a health-based incentive program can affect aspects of the insurance plan for an individual patient 3000. The claims database 5500 stores claim data 5510 on insurance claims submitted by the patient 3000 under the relevant insurance plan stored in the plan data 5310. The insurer computing system 5000 outputs the plan data 5310, program data 5410, and claim data 5510 under control of the processor 5100 for each individual patient 3000 to the health information system 200.

The health information system 200, as shown in FIGS. 1 and 2, receives an accumulated health data 2000 for each individual patient 3000 including the individual data 3010, the provider data 4010, the plan data 5310, the program data 5410, and the claim data 5510. The health information system 200 receives the accumulated health data 2000 continuously. The accumulated health data 2000 is all available forms of health data corresponding to each patient 3000 of the patient population 300. The accumulated health data 2000 received by the health information system 200 includes, for example, height, weight, blood pressure, known diseases, prescribed medications, filled prescriptions, adherence to prescribed medications, hospital records, physician records, compliance with preventative care screenings, condition specific monitoring studies, predisposition to diseases, lifestyle habits including smoking, usage of alcohol, exercise, diet, and all other forms of available data pertaining to individual health.

The accumulated health data 2000 received by the health information system 200 is stored in a behavior database 210 shown in FIG. 1 ordered by individual patient 3000. As described above, each health information system 200-1, 200-2 . . . 200-N may be a different entity ranging from a healthcare network to an employer, and consequently, each may have access to a different quantity and different range of health data. Each behavior database 210-1, 210-2 . . . 210-N therefore may contain a different quantity and different range of accumulated health data 2000 pertaining to each individual patient 3000 of the corresponding population 300-1, 300-2 . . . 300-N.

Each health information system 200, as shown in FIG. 1, also has an analysis unit 220, a communication module 230, an index database 240, a medical database 250, and a threshold database 260 connected with one another and with the behavior database 210. The communication module 230 may be any type of wired or wireless computing communication device known to those with ordinary skill in the art. The medical database 250 stores data on available medical treatments 4020 from the records database 4300 of the providers 400, available plans from the plan data 5310 of the insurers 500, and available programs from the program data 5410 of the insurers 500 related to the health information system 200. The medical database 250 also stores general medical information 6000. The threshold database 260 stores score thresholds 262, appointment thresholds 264, and insurer thresholds 266 described in greater detail below.

Each health information system 200-1, 200-2 . . . 200-N transmits the accumulated health data 2000 from the behavior database 210 to a behavior unit 160 of the health index system 100.

The health index system 100 calculates a health index 136, a data quality indicator 142, and a patient activation indicator 152 based on the accumulated health data 2000 for each patient 3000 in each patient population 300, as will now be described with reference to FIGS. 1 and 3-8.

The health index system 100, as shown in FIG. 1, includes a processor 110, a memory 120, the behavior unit 160, an output unit 170, and an index database 180. The health index system 100 is a system separate from the health information systems 200-1 . . . 200-N and may be positioned remotely from the health information systems 200-1 . . . 200-N. The memory 120 is a non-transitory computer readable medium capable of storing program instructions executable by the processor 110. The memory 120 has stored thereon an index unit 130, a data quality unit 140, and a patient activation unit 150. The index unit 130 includes a plurality of subcomponent calculation algorithms 132 and an index weighting algorithm 134.

The calculation of the health index 136 for each patient 3000 will now be described with reference to FIGS. 1 and 3-7.

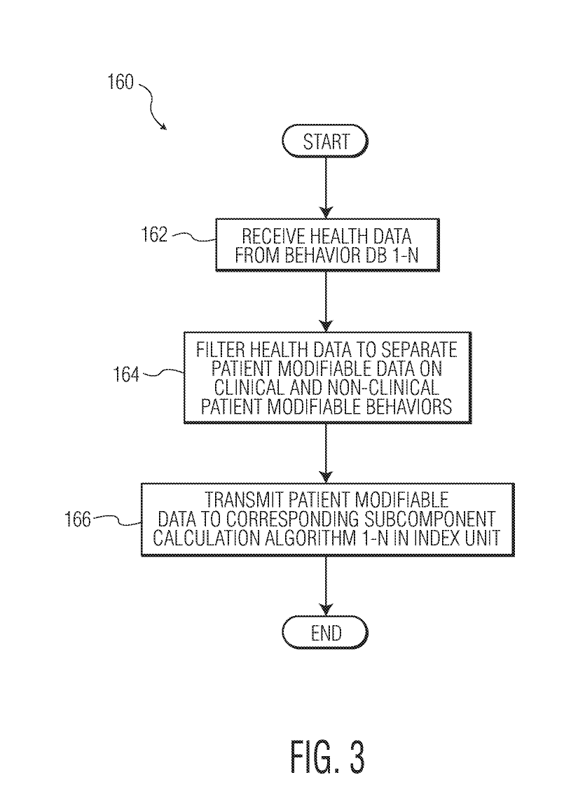

A process performed by the behavior unit 160 under the control of the processor 110 is shown in FIG. 3. The behavior unit 160 first receives the accumulated health data 2000 from the behavior database 210 of a health information system 200 in step 162. In an embodiment, the behavior unit 160 is a computing device capable of communicating with each health information system 200, such as by a wired connection or any wireless connection known to those with ordinary skill in the art, and also has a filtering algorithm stored thereon in a non-transitory computer readable medium executable by the processor 110. While receiving the accumulated health data 2000 in step 162, the behavior unit 160, performing data processing known to those with ordinary skill in the art, processes the accumulated health data 2000 to transform the data in disparate formats from disparate sources each into a unified format for further processing.

The filtering algorithm of the behavior unit 160 is executed by the processor 110 to filter the accumulated health data 2000 for each patient 3000 in step 164. The filtering step 164 separates data that relates to clinical and non-clinical patient modifiable behaviors, referred to herein as patient modifiable data 2010 from a remainder of the accumulated health data 2000. The patient modifiable data 2010 is defined as including health data relating only to those behaviors that the patient 3000 can choose or not choose to perform. The behaviors may be positive or negative. The patient modifiable data 2010 can include data on clinical behaviors related to clinical medical treatment, such as consistently taking medication as prescribed and participating in preventative screenings, and non-clinical behaviors, such as smoking and exercise. The patient modifiable data 2010 does not include data related to behaviors that are not modifiable by the patient 3000; for example, existing medical conditions and hereditary predispositions. In an exemplary embodiment, the fact that a patient 3000 has the medical condition of hypertension would not be included in the patient modifiable data 2010 but the adherence of the patient 3000 to taking antihypertensive medication would be included in the patient modifiable data 2010.

After filtering the accumulated health data 2000 in step 164, as shown in FIG. 3, in step 166 the processor 110 transmits the patient modifiable data 2010 filtered at the behavior unit 160 to a corresponding subcomponent calculation algorithm 132 shown in FIG. 1.

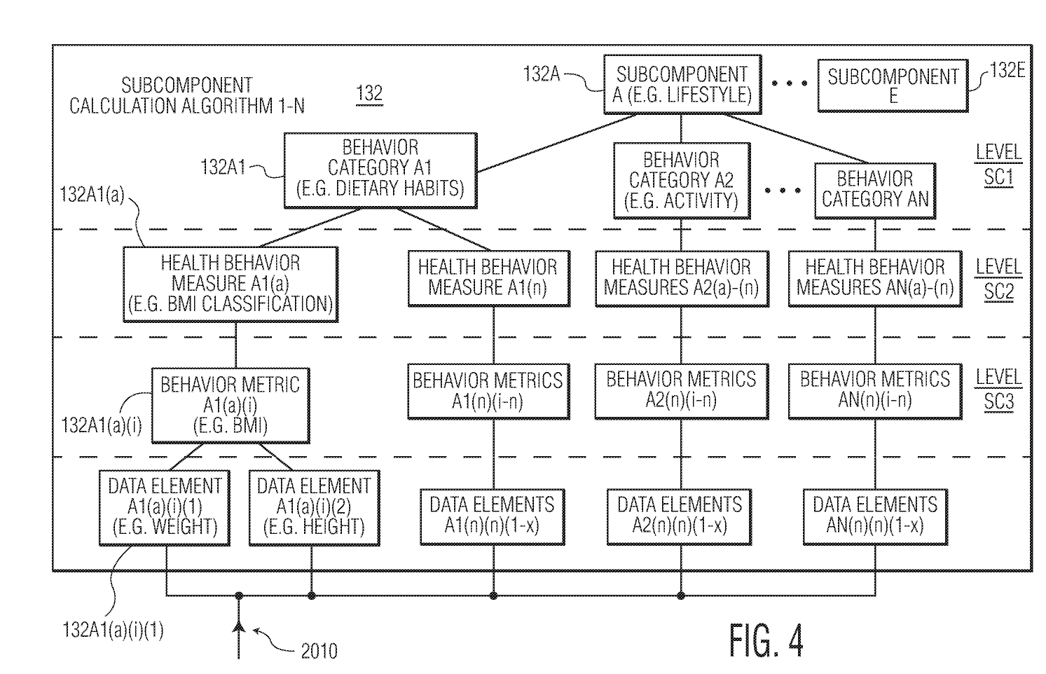

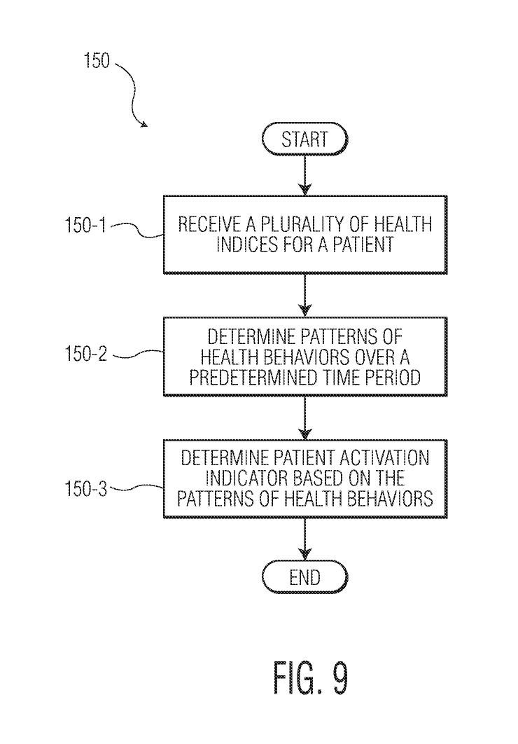

The health index 136 for each patient 3000 is calculated based on a separation of the patient modifiable data 2010 into a plurality of subcomponents 132A-E, shown in FIG. 4, which are each given a subcomponent score 132A-E(S) based on a separate subcomponent calculation algorithm 132 particular to that subcomponent 132A-E. In the embodiment shown in FIG. 9, the subcomponents 132A-E include a Life Style subcomponent 132A, a Wellness and Preventative Care subcomponent 132B, a Service Utilization subcomponent 132C, a Disease Maintenance subcomponent 132D, and a Medication Therapies subcomponent 132E. The shown subcomponents are merely exemplary and, in other embodiments, the health index 136 may be calculated based on additional or other subcomponents.

Each of the subcomponents 132A-E is calculated by the subcomponent calculation algorithm 132 of the index unit 130 based on sub-category levels SC1-SC3 including increasingly granular data pertaining to the subcomponents 132A-E. The calculation of the Life Style subcomponent 132A will now be described by way of example with reference to FIGS. 4-6 but applies equally to each of the various subcomponents 132A-E, varying only by the data that comprises each particular subcomponent 132A-E.

For the Life Style subcomponent 132A shown in FIG. 4, the subcomponent 132A includes a plurality of behavior categories 132A1-N at level SC1 including at least Dietary Habits 132A1 and Activity 132A2. The behavior category Dietary Habits 132A1 includes, for example, a plurality of health behavior measures A1(a)-(n) at level SC2 including a BMI classification A1(a) for the patient 3000. The BMI classification A1(a) at level SC2 is calculated based on a behavior metric A1(a)(i) at level SC3, in this example, a BMI of the patient 3000 as shown in FIG. 4. The BMI of the patient 3000 at level SC3 is calculated based on the data elements A1(a)(i)(1) of the patient's weight and A1(a)(i)(2) of the patient's height which were included in the patient modifiable data 2010 from the behavior unit 160 and are separated by the subcomponent calculation algorithm 132 particular to the Life Style subcomponent 132A.

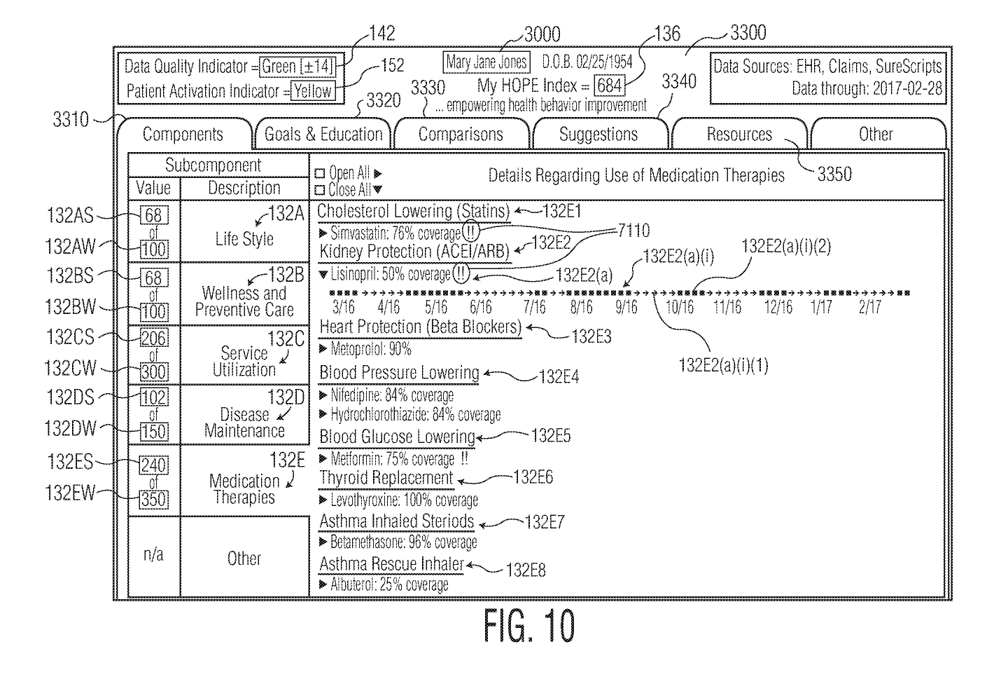

A similar hierarchy of the levels SC1-SC3 of subcategories for each subcomponent 132A-E including the behavior categories of SC1, the health behavior measures of SC2, the behavior metrics of SC3, and the data elements from the patient modifiable data 2010 is created for each subcomponent 132A-E; in another exemplary embodiment for the Medication Therapies subcomponent 132E shown in FIG. 10, each of the behavior categories E1-E8 is the adherence of the patient 3000 to taking one particular type of prescribed medication. As further shown in FIG. 9, the behavior category Kidney Protection 132E2 has a health behavior measure of usage of the medication lisinopril 132E2(a). The health behavior measure of the lisinopril 132E2(a) is calculated by the behavior metric of percentage of coverage of lisinopril 132E2(a)(i), calculated from data elements including time periods 132E2(a)(i)(1) and instances of filling lisinopril prescriptions in the time periods 132E2(a)(i)(2).

The processor 110 executes the subcomponent calculation algorithm 132 in the index unit 130 for each subcomponent 132A-E to calculate a subcomponent score 132A-E(S) for each subcomponent 132A-E. The subcomponent score 132A-E(S) for a subcomponent 132A-E is a weighted sum of the scores for each behavior category at level SC1 in FIG. 4. In an exemplary embodiment, the subcomponent score 132AS for the Life Style subcomponent 132A is an integer between 1 and 1000; each of the behavior categories 132A1-N at level SC1 has an integer score between 1 and 1000 with a corresponding weighted percentage, and likewise, each of the behavior metrics 132A1(a)(i-n) at level SC2 has an integer score between 1 and 1000 and a corresponding weighted percentage. At each level SC1 and SC2, each behavior category and health behavior measure has a score between 1 and 1000 and a weighted percentage with the total weighted percentage at each level SC1 and SC2 adding up to 100%.

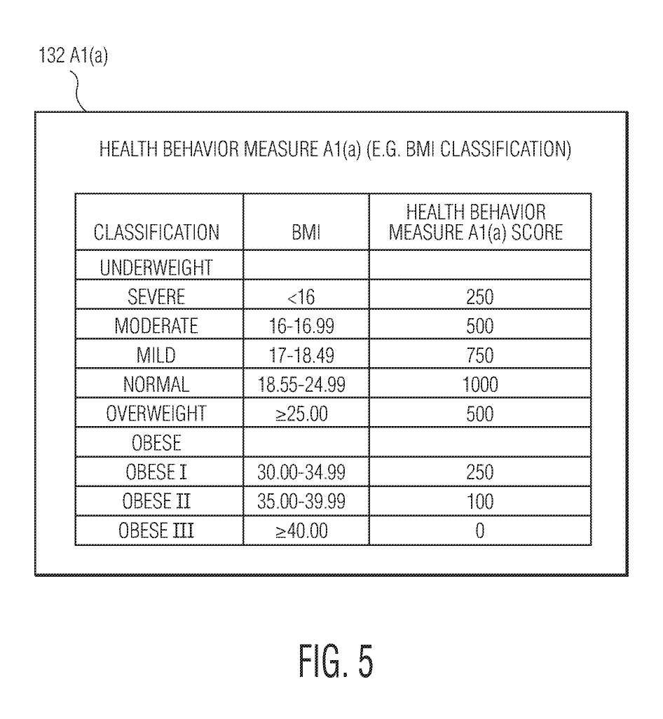

In an exemplary embodiment shown in FIGS. 4 and 5, to determine the score for the Dietary Habits behavior category 132A1 of the Life Style subcomponent 132A, the BMI classification health behavior measure 132A1(a) is determined by the processor 110. The processor 110 executes the index unit 130 to compare the BMI behavior metric 132A1(a)(i) for a patient 3000 to a classification chart shown in FIG. 5, which is stored as the health behavior measure 132A1(a) in the subcomponent calculation algorithm 132. The score for the health behavior measure 132A1(a) is determined based on the chart. The scores for each health behavior measure 132(A)(a-n) contributing to the Dietary Habits behavior category 132A1 are similarly determined and are then weighted and added together to determine the score for the Dietary Habits behavior category 132A1.

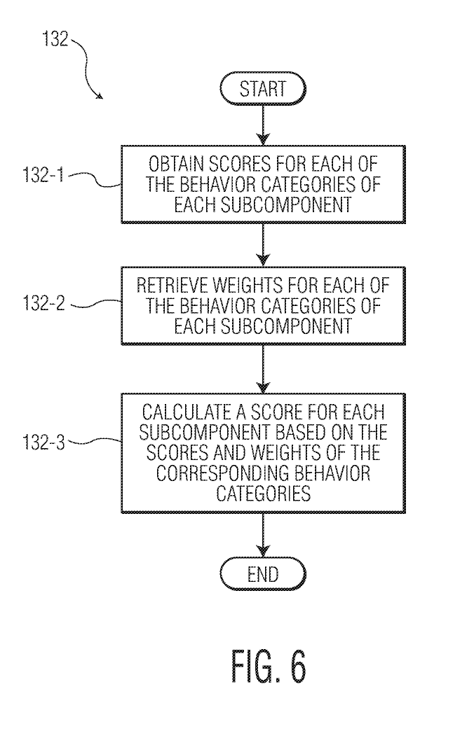

A process to determine the subcomponent score 132A-E(S) for each subcomponent 132A-E is shown in FIG. 6. The process is performed by the processor 110 executing the subcomponent calculation algorithm 132. The processor 110 first executes the subcomponent calculation algorithm 132 in a first step 132-1 to obtain the scores for each of the behavior categories of each subcomponent at level SC1, as exemplarily described above for the Dietary Habits behavior category 132A1. In a second step 132-2, the processor 110 retrieves weights stored in the subcomponent calculation algorithm 132 for each of the behavior categories of each subcomponent. In an embodiment, the weights stored within the subcomponent calculation algorithm 132 at each of the levels SC1, SC2, and SC3 are adjustable based, for example, on an individual patient's 3000 health or on changes in medical knowledge. In a final step 132-3, the processor 110 executes the subcomponent calculation algorithm 132 to calculate a score 132A-E(S) for each subcomponent 132A-E based on the scores and weight of the corresponding behavior categories.

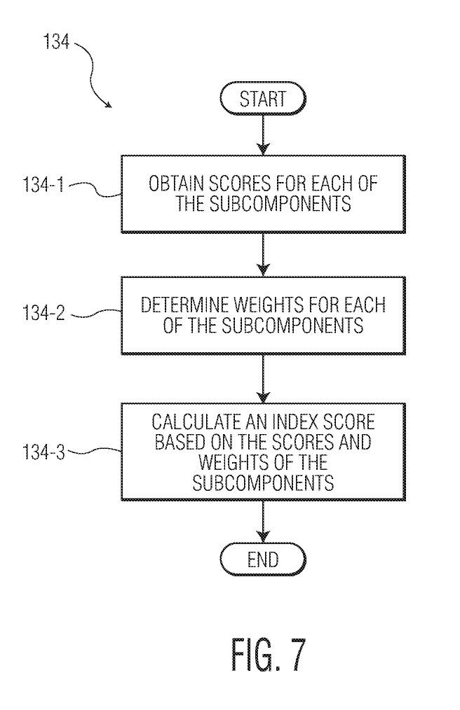

A process to determine the health index 136 for the patient 3000 is shown in FIG. 7. The process is performed by the processor 110 executing the index weighting algorithm 134. The processor 110 in a first step 134-1 executes the index weighting algorithm 134 to obtain subcomponent scores 132A-E(S) for each of the subcomponents 132A-E calculated at the subcomponent calculation algorithms 132. In a second step 134-2, the processor 110 retrieves weights 132A-E(W) stored in the index weighting algorithm 134 for each of the subcomponent 132A-E. In an embodiment, the weights 132A-E(W) stored within the index weighting algorithm 132 are adjustable based, for example, on an individual patient's 3000 health or on changes in medical knowledge. In a final step 134-3, the processor 110 executes the index weighting algorithm 134 to calculate the health index 136 based on the scores and weights of the subcomponents 132A-E; as similarly described above, the score 132A-E(S) for each subcomponent 132A-E is multiplied by the weight 132A-E(W) for the subcomponent 132A-E and the weighted subcomponent 132A-E scores are added together to determine the health index 136 for the patient 3000.

In an embodiment, the health index 136 is a single numerical value between 1 and 1000. An embodiment of the subcomponent scores 132A-E(S) and weight 132A-E(W) is shown in FIG. 10. The index unit 130, under control of the processor 110, stores the calculated health index 136 in the index database 180 ordered by patient 3000; the index database 180 is capable of storing a plurality of health indices 136 calculated over time for each patient 3000.

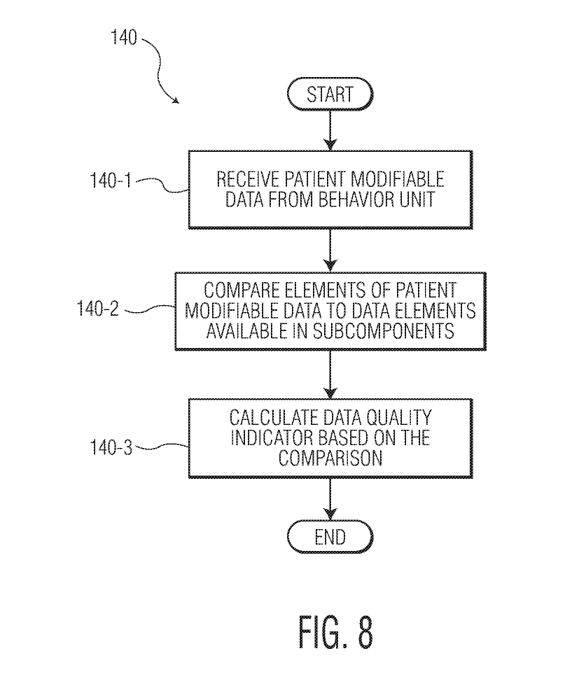

The health index system 100 also calculates the data quality indicator 142 which, in certain embodiments, accompanies the health index 136 for the patient 3000. The processor 110 executes an algorithm stored in the data quality unit 140 to perform a process shown in FIG. 8 determining the data quality indicator 142. In a first step 140-1, the data quality unit 140 receives the patient modifiable data 2010 from the behavior unit 160; as described above, the patient modifiable data 2010 includes filtered data from the individual data 3010, the provider data 4010, the plan data 5310, the program data 5410, and the claim data 5510. In a next step 140-2, the data quality unit 140 compares the elements of data received in the patient modifiable data 2010 to all data elements which are capable of being incorporated into a subcomponent 132A-E calculation by the subcomponent calculation algorithm 132, as shown in the exemplary embodiment of FIG. 4.

In a final step 140-3 shown in FIG. 8, the data quality unit 140 calculates the data quality indicator 142 based on the comparison. As described above, each health information system 200 may vary in scope and, consequently, the data available to calculate the health index 136 for the patients 3000 of various health information systems 200 may vary; the data quality indicator 142 is a relative measure of the breadth of data incorporated into the calculation of the health index 136 for an individual patient 3000. In an embodiment shown in FIG. 10, the data quality indicator 142 is represented by a color or word ranging from red to orange to yellow to green, with green representing robust available data and red representing minimal available data. The data quality indicator 142, as shown in FIG. 10, may also be represented by a numerical confidence interval for the health index 136 in conjunction with the color or word.

The health index system 100 also calculates the patient activation indicator 152 which, in certain embodiments, accompanies the health index 136 and data quality indicator 142 for the patient 3000. The processor 110 executes an algorithm stored in the patient activation unit 150 to perform a process shown in FIG. 9 determining the patient activation indicator 152. In a first step 150-1, the patient activation unit 150 receives a plurality of health indices 136 calculated for the patient 3000 over time from the index database 180. In a next step 150-2, the patient activation unit 150 determines patterns of health behaviors in the patient modifiable data 2010 of the plurality of health indices 136 over a predetermined time period. The patterns of health behaviors include patterns ranging from a pattern of health behavior associated with a high level of patient 3000 engagement, suggesting that the patient 3000 is commonly active in improving his or her own health, to a pattern of health behavior associated with a low level of patient 3000 engagement, suggesting that the patient 3000 is commonly inactive in improving his or her own health.

In a final step 150-3 shown in FIG. 9, the patient activation unit 150 determines the patient activation indicator 152 based on the patterns of health behaviors. In an embodiment shown in FIG. 10, the patient activation indicator 152 is represented by a color or word ranging from red to orange to yellow to green, with green representing a marked increase in the health indices 136 over time and red representing a marked decrease in the health indices 136 over time. In other embodiments, the patient activation indicator 152 may be a numerical value.

The health index system 100 outputs the health index 136 and the data quality indicator 142 for each patient 3000 in each patient population 300, as will be described in greater detail below with reference to FIGS. 1, 2, and 10-16.

The processor 110 transmits the health index 136 at the index unit 130, the data quality indicator 142 at the data quality unit 140, the patient activation indicator 152 at the patient activation unit 150, and the patient modifiable data 2010 to the output unit 170, shown in FIG. 1. The processor 110 controls the output unit 170 to output the health indices 136, data quality indicators 142, patient activation indicators 152, and patient modifiable data 2010 for each patient 3000 in each patient population 300 to the respective health information system 200. In an embodiment, the output unit 170 is a computing device capable of communicating with each health information system 200 by a wired connection or any wireless connection known to those with ordinary skill in the art under control of the processor 110.

The health information system 200 receives the health indices 136, data quality indicators 142, patient activation indicators 152, and patient modifiable data 2010 and stores these at the index database 240 of the health information system 200, shown in FIG. 1, ordered by patient 3000. In various embodiments, the health information system 200 may transmit the patient accumulated data 2000 to the health index system 100 continuously or on request of the health information system 200; likewise, the health information system 200 may receive the health indices 136, data quality indicators 142, patient activation indicators 152, and patient modifiable data 2010 from the health index system 100 continuously or on request of the health information system 200. In an embodiment, the health information system 200 stores the health indices 136 ordered by provider 400 to monitor how providers 400 correspond to positive health behaviors of patients 3000.

Each patient 3000 in the patient population 300 can access his or her health index 136 at the health information system 200 via the communication module 230 controlled by the analysis unit 220. As similarly described for other components above and shown in FIG. 1, the analysis unit 220 includes a processor 222 and a memory 224. The memory 224 is a non-transitory computer readable medium storing algorithms executable by the processor 222 of the analysis unit 220 to perform the processes and functions of the analysis unit 220 described herein.

The communication module 230 receives the request from the patient device 3200 and, under the control of the processor 222 of the analysis unit 220, retrieves the health index 136 from the index database 240 and transmits the health index 136 associated with the particular patient 3000 to the patient device 3200. The processor 3210 of the patient device 3200 incorporates the health index 136 into a patient interface 3300 stored on the memory 3220 and outputs the patient interface 3300 to the display interface 3230 of the patient device 3200. The display interface 3230 of the patient device 3200 may be any type of electronic device display known to those with ordinary skill in the art capable of displaying information and receiving either a direct contact input or an indirect signal input. The data quality indicator 142, patient activation indicator 152, patient modifiable data 2010, and information from the medical database 250 can be similarly retrieved and incorporated into the patient interface 3300.

An exemplary patient interface 3300 displayed on the display interface 3230 of the patient device 3200 is shown in FIGS. 10-14. In the shown embodiment, the patient interface 3300 always shows the health index 136, data quality indicator 142, patient activation indicator 152, and the subcomponents 132A-E including the subcomponent scores 132A-E(S) and subcomponent weights 132A-E(W). The patient interface 3300 also has a number of tabs 3310-3350 which permit the patient 3000 to switch between various data displayed adjacent to the health index 136, data quality indicator 142, patient activation indicator 152, and the subcomponents 132A-E.

In a components tab 3310 shown in FIG. 10, the patient 3000 has access to the data that comprises the previously described calculation of each subcomponent 132A-E. In the exemplary embodiment shown in FIG. 10, the patient 3000 may select the Medication Therapies subcomponent 132E and, under the control of the processor 3210, view the eight medication behavior categories 132E1-8 contributing to the Medication Therapies score 132ES. The patient 3000 can further interact with the components tab 3310 via the display interface 3230 to examiner further levels SC2, SC3 of the subcomponent 132A-E as shown in FIG. 4. In the exemplary embodiment shown in FIG. 10, selection of the behavior category Kidney Protection 132E2 shows the health behavior measure of usage of lisinopril 132E2(a) including the behavior metric of percentage of coverage of lisinopril 132E2(a)(i) and the data elements including time periods 132E2(a)(i)(1) and instances of taking lisinopril in the time periods 132E2(a)(i)(2). The components tab 3310 functions similarly for the behavior categories of each subcomponent 132A-E and permits the patient 3000 to examine all levels SC1-SC2 contributing to the health index 136 down to the data elements of the patient modifiable data 2010.

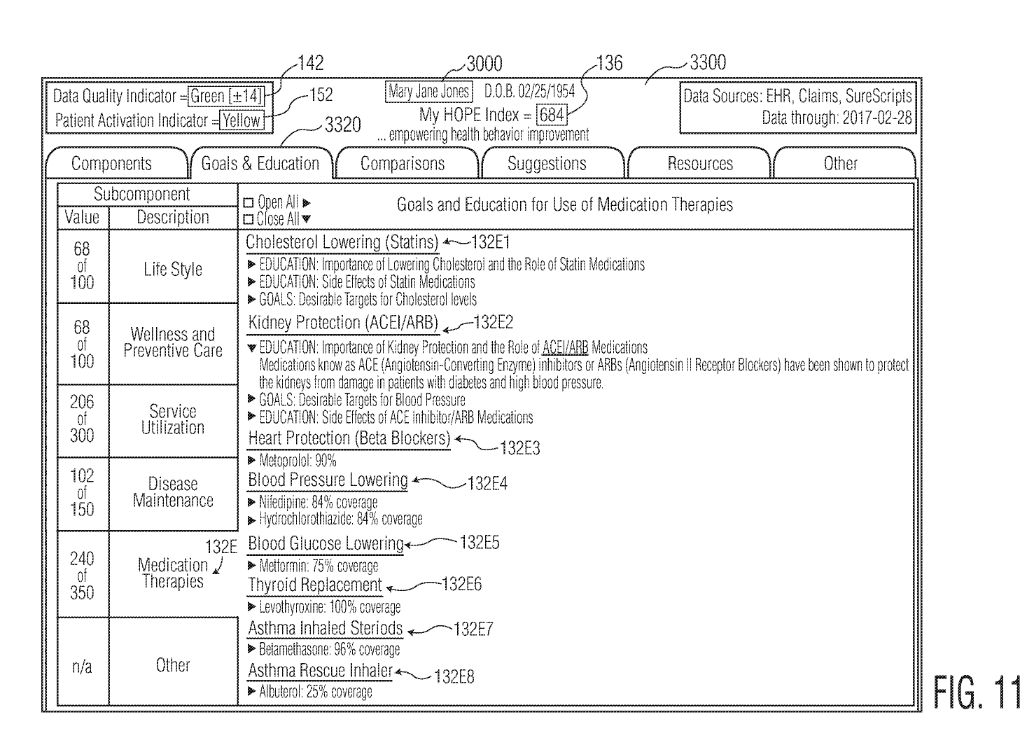

In a goals and education tab 3320 shown in FIG. 11, the patient interface 3300 displays goals and medical information from the general medical information 6000 stored on the medical database 250. The goals and medical information are ordered by behavior category; in the embodiment shown in FIG. 11, the goals and medical information are particular to the medications and goals of treatment in each medication behavior category 132E1-8 of the Medication Therapies subcomponent 132E. As would be understood by one with ordinary skill in the art, similar goals and medical information are available for each subcomponent 132A-E and the patient 3000 can access these through the patient interface 3300.

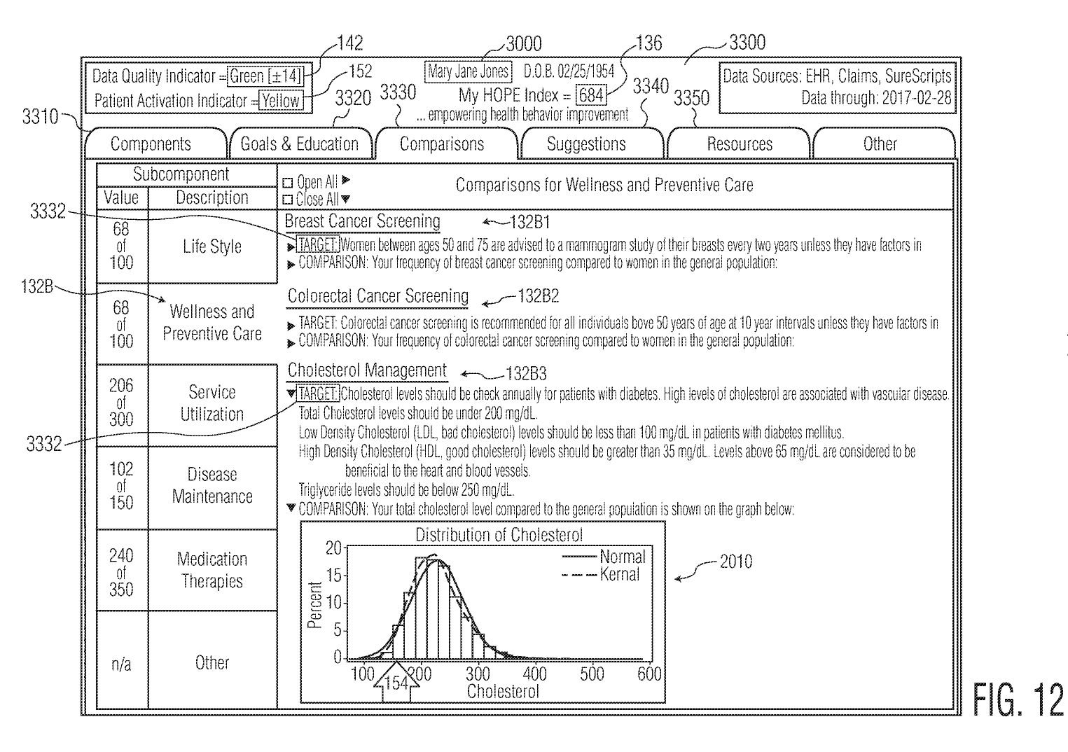

In a comparisons tab 3330 shown in FIG. 12, the patient interface 3300 displays a target 3332 and comparison of a portion of the patient's 3000 patient modifiable data 2010 to the target 3332 for each behavior category of the subcomponent 132A-E. The target 3332 is retrieved from the general medical information 6000 and the processor 3210 executes the comparison for display on the patient interface 3300. In the embodiment shown in FIG. 12, the Wellness and Preventative Care subcomponent 132B is selected and the patient's 3000 cholesterol from the patient modifiable data 2010 is compared to the target 3332 for the Cholesterol Management behavior category 132B3. As would be understood by one with ordinary skill in the art, similar targets and comparisons are available for each subcomponent 132A-E and the patient 3000 can access these through the patient interface 3300.

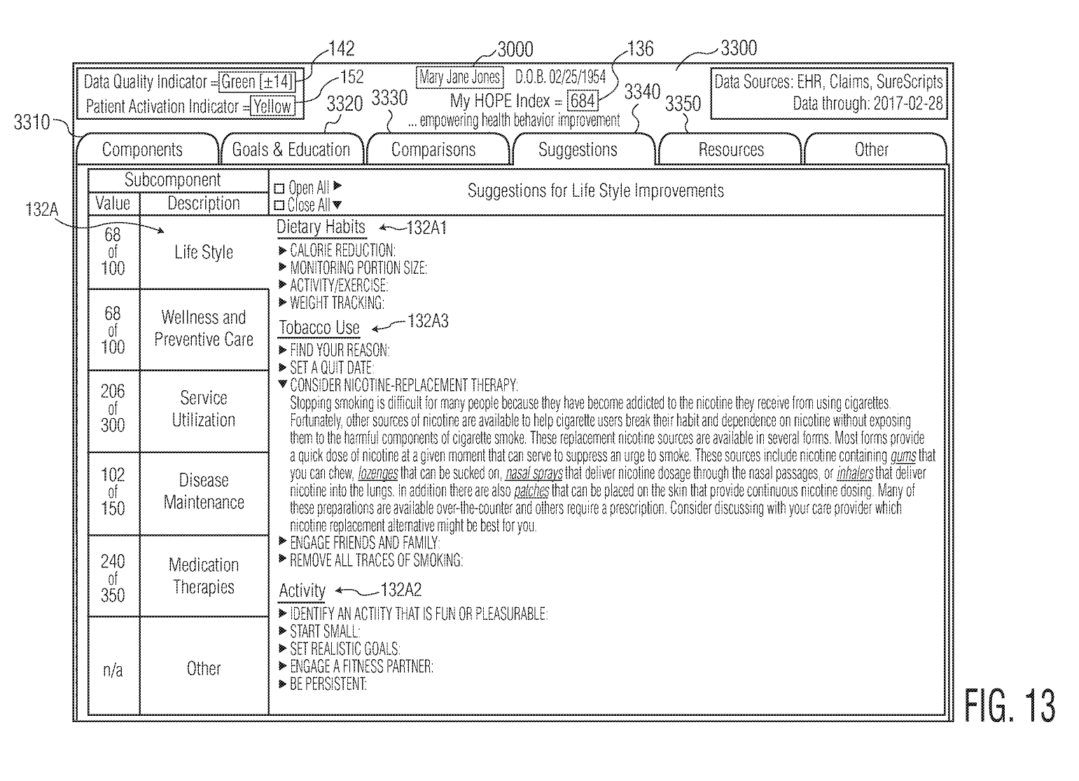

In a suggestions tab 3340 shown in FIG. 13, the patient interface 3300 displays suggestions for addressing each behavior category of the subcomponent 132A-E. The suggestions are retrieved from the general medical information 6000 or the program data 5410 stored in the medical database 250. In the embodiment shown in FIG. 13, the Life Style subcomponent 132A lists a suggestion for the Tobacco Use behavior category 132A3. As would be understood by one with ordinary skill in the art, similar suggestions are available for each subcomponent 132A-E and the patient 3000 can access these through the patient interface 3300.

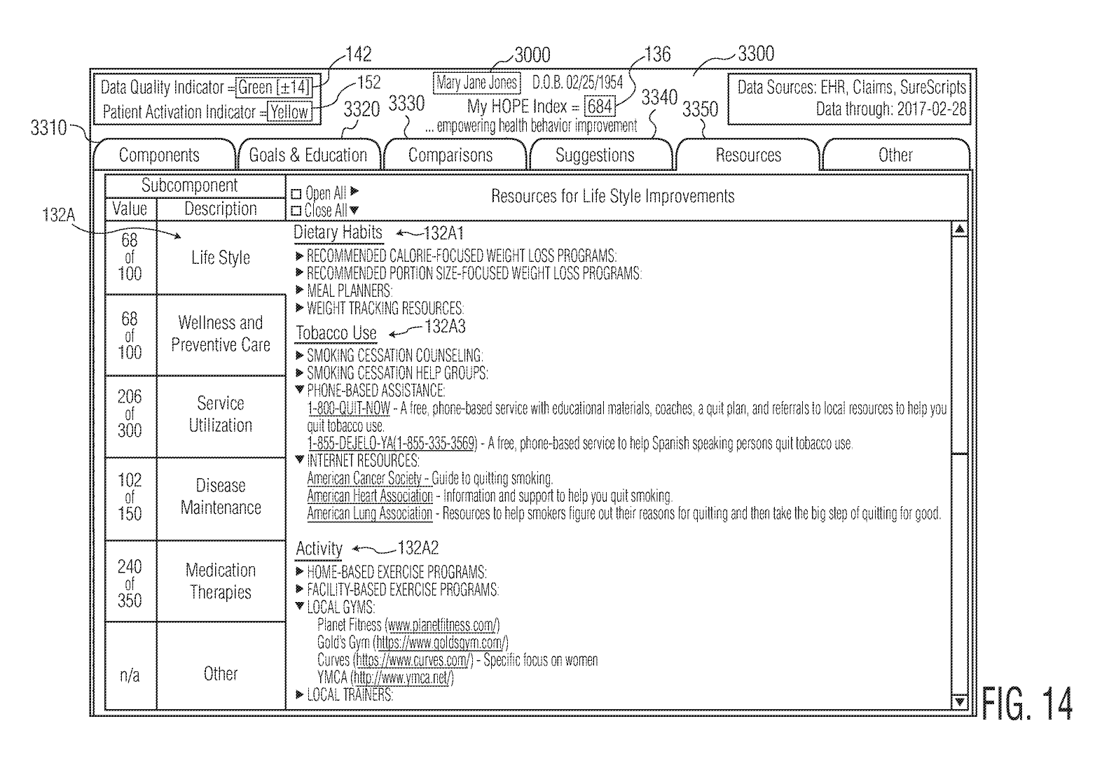

In a resources tab 3350 shown in FIG. 14, the patient interface 3300 displays resources available to address each behavior category of the subcomponent 132A-E. The resources are retrieved from the available medical treatments 4020, the program data 5410, and the general medical information 6000. In the embodiment shown in FIG. 14, the Life Style subcomponent 132A has detailed resources for each of the Activity 13A2 and Tobacco Use 132A3 behavior categories. As would be understood by one with ordinary skill in the art, similar resources are available for each subcomponent 132A-E and the patient 3000 can access these through the patient interface 3300.

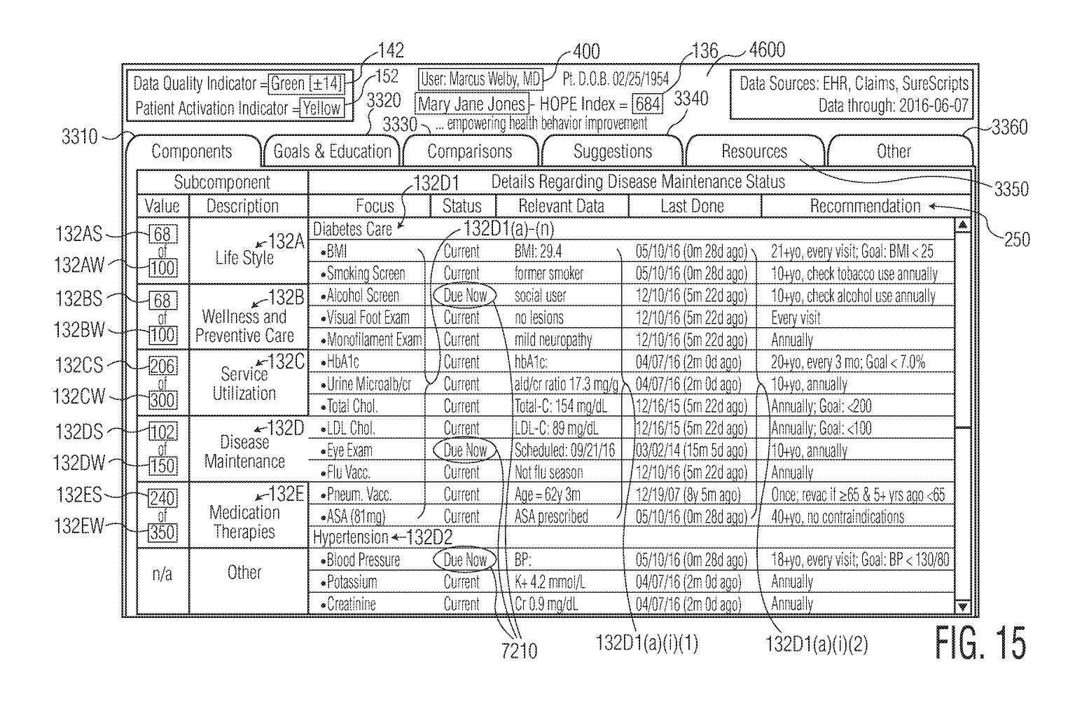

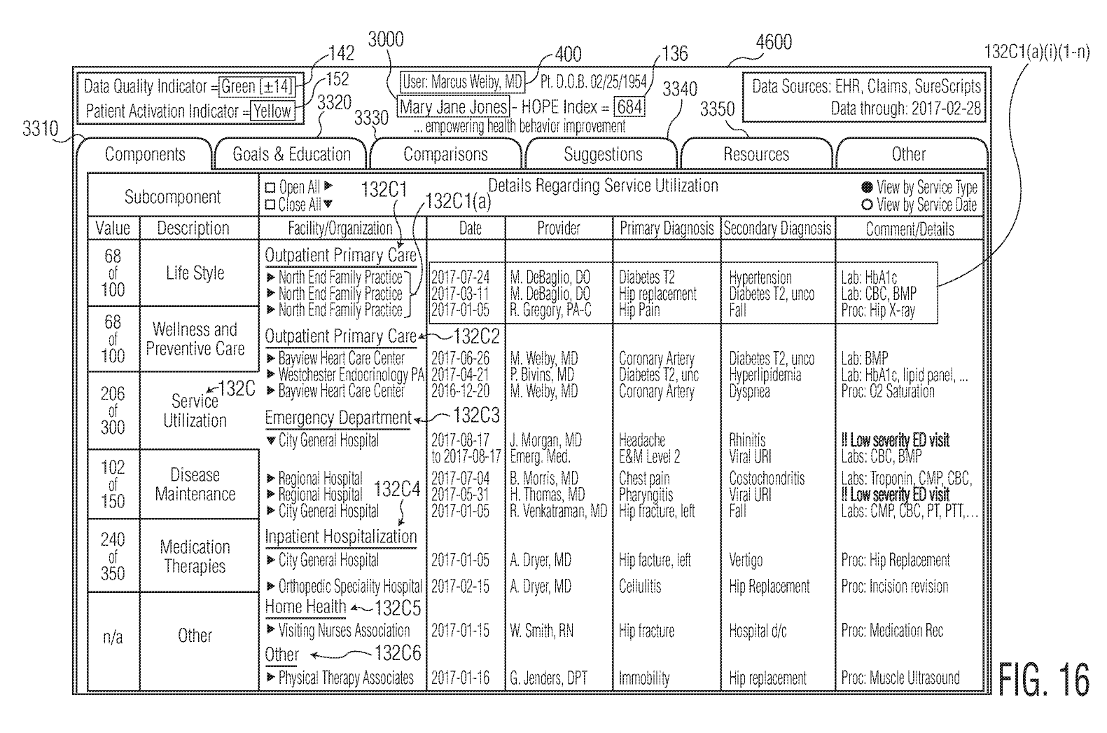

In an embodiment, each provider 400 can similarly access the health index 136 for each patient 3000 of the provider 400 at the provider computing system 4000. The communication module 230 of the health information system 200 receives the request from the provider computing system 4000, retrieves the health index 136 from the index database 240, and transmits the health index 136 to the provider computing system 4000. The processor 4100 of the provider computing system 4000 incorporates the health index 136 into a provider interface 4600 shown in FIGS. 15 and 16 stored on the memory 4200 and outputs the provider interface 4600 to the display interface 4400 of the provider computing system 4000. The display interface 4400 of the provider computing system 4000 may be any type of electronic device display known to those with ordinary skill in the art capable of displaying information and receiving either a direct contact input or an indirect signal input. The data quality indicator 142, patient modifiable data 2010, and information from the medical database 250 can be similarly retrieved and incorporated into the provider interface 4600.

The provider interface 4600 is similar to the patient interface 3300 described above and all tabs 3310-3350 are capable of displaying the same range of information described above with reference to FIGS. 10-14. The provider interface 4600, as shown in FIGS. 15 and 16, always displays the provider 400 in addition to the health index 136, data quality indicator 142, and the subcomponents 132A-E including the subcomponent scores 132A-E(S) and subcomponent weights 132A-E(W) for the patient 3000. In the exemplary embodiments shown in FIGS. 15 and 16, the provider 400 has selected the components tab 3310. In FIG. 15, the provider 400 has selected the Disease Maintenance subcomponent 132D and, under the control of the processor 4100, can view the behavior categories of Diabetes Care 132D1 and Hypertension 132D2 along with the data elements 132D1(a)(i)(1-n) contributing to the behavior categories. In FIG. 16, the provider 400 has selected the Service Utilization subcomponent 132C and, under the control of the processor 4100, can view the behavior categories 132C1-C6 of various types of medical services used by the patient 3000. The Outpatient Primary Care subcomponent 132C1 is displayed, for example, along with health behavior measures 132C1(a) of instances of visits to a particular practice and data elements 132C1(a)(i)(1-n) relevant to those health behavior measures 132C1(a). In other embodiments, the provider 400 can use the provider interface 4600 to track trends in health indices 136 for patients 3000.

Each insurer 500 is not shown with a computing system in the described embodiments, however, one with ordinary skill in the art would understand that the each insurer 500 could also have a display interface in some embodiments displaying the tabs 3310-3350 and information described above in the patient interface 3300 and provider interface 4600. In some embodiments, the display interface for the insurer 500 may differ from that of the patient interface 3300 and provider interface 4600. The insurer 500 display interface may focus on particular areas of interest for the insurer 500, for example, categorizing the patient modifiable data 2010 from most unfavorable behaviors to most favorable behaviors and further including projected costs of the unfavorable behaviors.

The healthcare resource system 1 determines and executes health actions 7000 based on information from the health index system 100 including executing score alerts 7100, appointment alerts 7200, and insurer actions 7300, as will be described in greater detail below with reference to FIGS. 1, 2, and 17-19. The analysis unit 220 of the health information system 200 determines the health actions 7000 and transmits the health actions 7000 to the patient 3000, provider 400, and/or insurers 500 which execute the health action 7000, as described below.

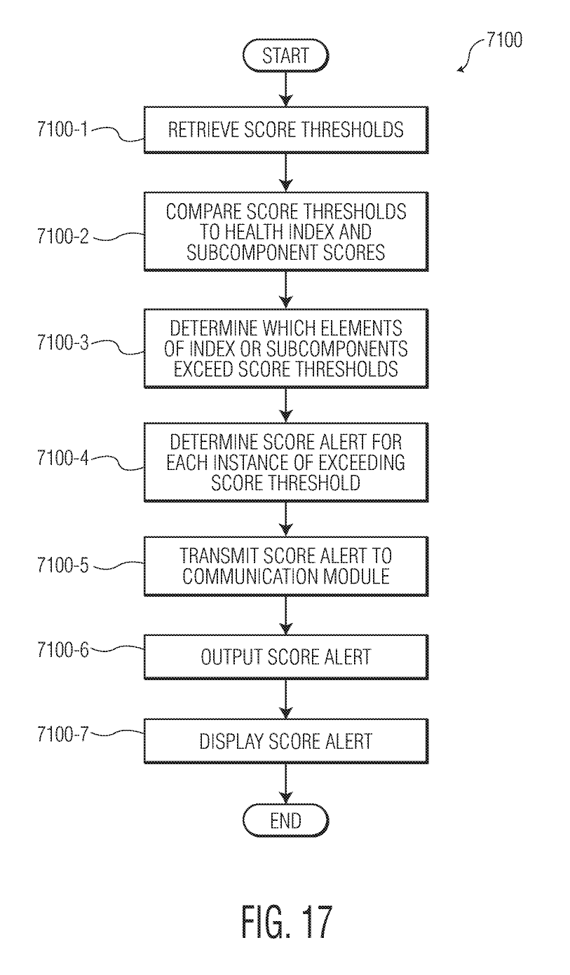

The execution of score alerts 7100 in the healthcare resource system 1 is shown in FIG. 17. In a first step 7100-1, the analysis unit 220 retrieves the score thresholds 262 stored in the threshold database 260. The score thresholds 262 correspond to the health index 136, subcomponents 132A-E comprising the health index 136, health behavior measures comprising the subcomponents 132A-E, and/or any other scores in the hierarchy of each subcomponent 132A-E shown in FIG. 4. In an embodiment, the score thresholds 262 are set by the providers 400 of the health information system 200. In another embodiment, the score thresholds 262 are set from the general medical information 6000 in the medical database 250. In various embodiments, the score thresholds 262 may either be universal to all patients 3000 in the patient population 300 or may be set to correspond to individual patients 3000 in the threshold database 260.

In a next step 7100-2 shown in FIG. 17, the analysis unit 220 compares the score thresholds 262 to the health index 136 and subcomponent 132A-E scores of the patient 3000. Based on the comparison in step 7100-2, the analysis unit 220 determines whether the health index 136 as a whole or which elements in the hierarchy of the subcomponents 132A-E exceeds the corresponding score threshold 262 in step 7100-3. The analysis unit 220 determines a score alert 7110 for each instance of an element of the health index 136 exceeding the score threshold 262 in step 7100-4. In step 7100-5, the analysis unit 220 transmits the score alerts 7110 to the communication module 230.

In step 7100-6 shown in FIG. 17, the communication module 230 outputs the score alerts 7110 to the patient device 3200 and/or the provider computing system 4000. The patient device 3200 receives the score alerts 7110 at the processor 3210 and, in step 7100-7, the processor 3210 incorporates the score alert 7110 into the patient interface 3300 and displays the score alert 7110 in the patient interface 3300 on the display interface 3230 of the patient device 3200. The provider computing system 4000 can likewise display the score alerts 7110 on the provider interface 4600 as described above. An embodiment of a score alert 7110 displayed on the patient interface 3300 is shown in FIG. 10. In this embodiment, the score alert 7110 notifies the patient 3000 that the patient's adherence to taking certain medications is lacking, which is lowering the Medication Therapies subcomponent score 132ES.

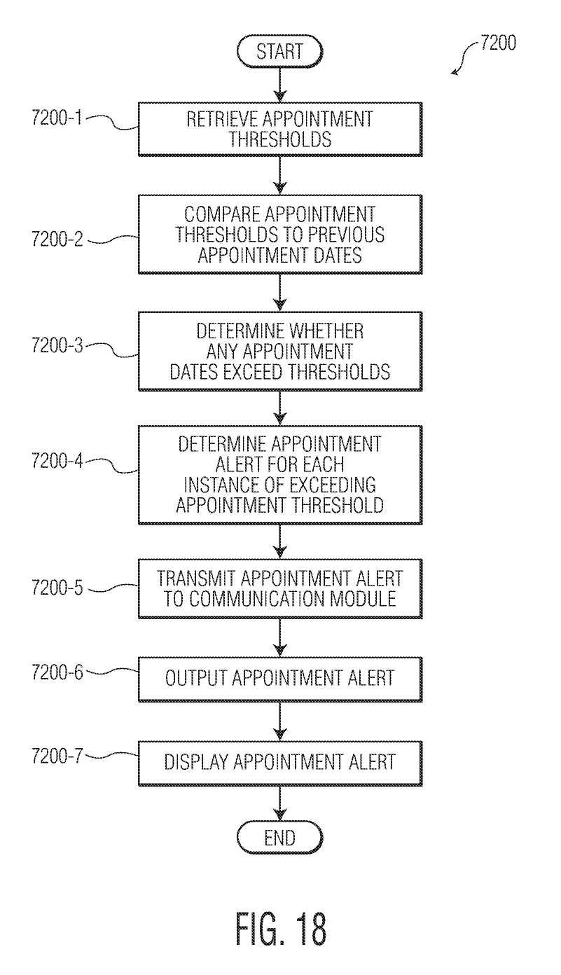

The execution of appointment alerts 7200 in the healthcare resource system 1 is shown in FIG. 18. In a first step 7200-1, the analysis unit 220 retrieves the appointment thresholds 264 stored in the threshold database 260. The appointment thresholds 264 are time period thresholds corresponding to previous appointments of the patient 3000 in the provider data 4010. In an embodiment, the appointment thresholds 264 are set by the providers 400 of the health information system 200. In another embodiment, the appointment thresholds 264 are set from the medical database 250. In various embodiments, the appointment thresholds 264 may either be universal to all patients 3000 in the patient population 300 or may be set to correspond to individual patients 3000 in the threshold database 260.

In a next step 7200-2 shown in FIG. 18, the analysis unit 220 compares the appointment thresholds 264 to the previous appointment dates in the provider data 4010. Based on the comparison in step 7200-2, the analysis unit 220 determines whether any appointment dates corresponding to any medical services of the providers 400 exceed the corresponding appointment threshold 264 in step 7200-3. The analysis unit 220 determines an appointment alert 7210 for each instance of a time period from a previous appointment date exceeding the appointment threshold 264 in step 7200-4. In step 7200-5, the analysis unit 220 transmits the appointment alerts 7210 to the communication module 230.

In step 7200-6 shown in FIG. 18, the communication module 230 outputs the appointment alert 7210 to the patient device 3200 and/or the provider computing system 4000. The patient device 3200 receives the appointment alerts 7210 at the processor 3210 and, in step 7200-7, the processor 3210 incorporates the appointment alert 7210 into the patient interface 3300 and displays the appointment alert 7210 in the patient interface 3300 on the display interface 3230 of the patient device 3200. The provider computing system 4000 can likewise display the appointment alerts 7210 on the provider interface 4600 as described above. An embodiment of an appointment alert 7210 displayed on the provider interface 4600 is shown in FIG. 15. In this embodiment, the appointment alert 7110 notifies the provider 400 that certain examinations and screens are due for the patient 3000, which are lowering the Disease Maintenance subcomponent score 132DS

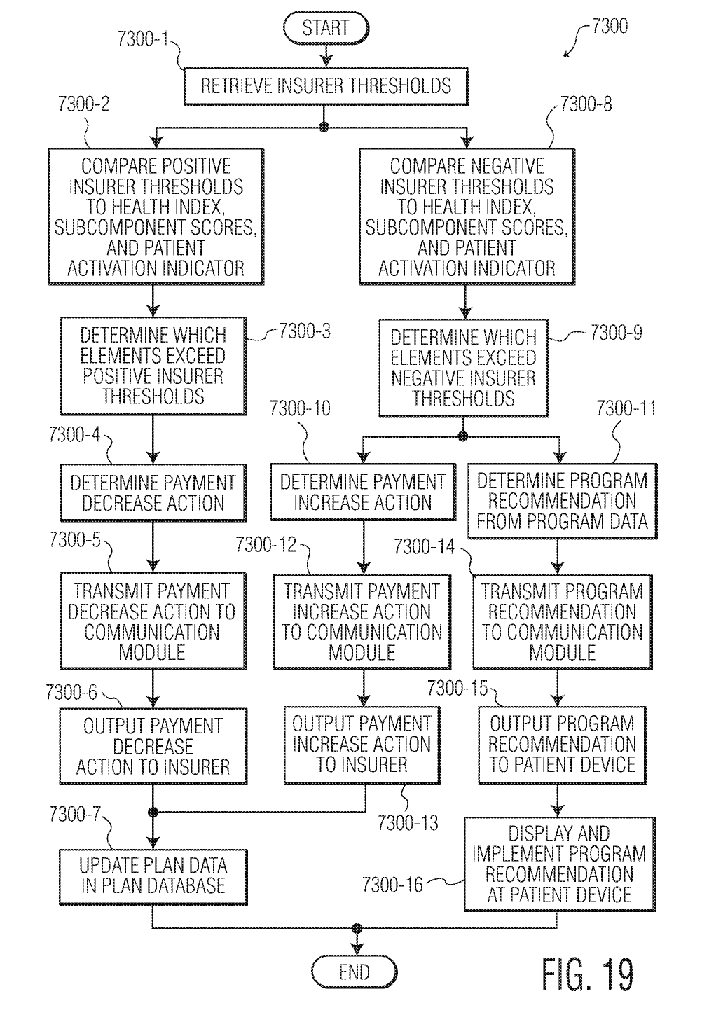

The execution of insurer actions 7300 in the healthcare resource system 1 is shown in FIG. 19. In a first step 7300-1, the analysis unit 220 retrieves the insurer thresholds 266 stored in the threshold database 260. In an embodiment, the insurer thresholds 266 are score thresholds corresponding to the health index 136, subcomponents 132A-E comprising the health index 136, health behavior measures comprising the subcomponents 132A-E, and/or any other scores in the hierarchy of each subcomponent 132A-E shown in FIG. 4. In another embodiment, the insurer thresholds 266 are score thresholds corresponding to the patient activation indicator 152. The insurer thresholds 266 are set by the insurers 500 and may be either universal to all patients 3000 in the patient population 300 or may be set to correspond to individual patients 3000 in the threshold database 260. The insurer thresholds 266 are divided into positive insurer thresholds 266A representative of healthy indications in the health index 136, subcomponents 132A-E, and/or patient activation indicator 152 and negative insurer thresholds 266B representative of unhealthy indications in the health index 136, subcomponents 132A-E, and/or patient activation indicator 152.

The analysis unit 220, as shown in FIG. 19, compares the positive insurer thresholds 266A to the health index 136, subcomponents 132A-E, and/or patient activation indicator 152 in step 7300-2. In a next step 7300-3, the analysis unit 220 determines which of the health index 136, subcomponents 132A-E, and/or patient activation indicator 152 exceeds positive insurer thresholds 266A and, in step 7300-4, determines a payment decrease action 7310 for each instance of the health index 136, subcomponent 132A-E, and/or patient activation indicator 152 exceeding a corresponding positive insurer threshold 266A. The analysis unit 220 transmits the payment decrease action 7310 to the communication module 230 in step 7300-5 and outputs the payment decrease action 7310 to the insurer computing system 5000 in step 7300-6.

The insurer computing system 5000 receives the payment decrease action 7310 at the processor 5100 and, in step 7300-7 shown in FIG. 19, the processor 5100 updates the plan data 5310 particular to the patient 3000 in the plan database 5300 with the payment decrease action 7310. In various embodiments, the payment decrease action 7310 may be a reduction in an insurance premium and/or a reduction in a copayment or some other form of reward. In an exemplary embodiment, the positive insurer threshold 266A is a fifty-point increase in the health index 136 in one year and meeting this threshold results in a 50% reduction in copayments in the plan data 5310 of the patient 3000. In another exemplary embodiment, the positive insurer threshold 266A is a health index 136 score of over 700 points and meeting this threshold results in a 10% reduction in insurance premiums in the plan data 5310 of the patient 3000.

In parallel with the positive insurer threshold 266A steps 7300-2 to 7300-7, the analysis unit 220 similarly performs negative insurer threshold steps 7300-8 to 7300-16 shown in FIG. 19. In step 7300-8, the analysis unit 220 compares the negative insurer thresholds 266B to the health index 136, subcomponents 132A-E, and/or patient activation indicator 152. In a next step 7300-9, the analysis unit 220 determines which of the health index 136, subcomponents 132A-E, and/or patient activation indicator 152 exceed negative insurer thresholds 266B. In steps 7300-10 and 7300-11, based on the determination in step 7300-9, the analysis unit 220 determines a payment increase action 7320 and/or a program recommendation 7330 for each instance of the health index 136, subcomponent 132A-E, and/or patient activation indicator 152 exceeding a corresponding negative insurer threshold 266B. The program recommendation 7330 is from the program data 5410 of the insurer 500 and is a health-based incentive program corresponding to the health issue raised by exceeding the negative insurer threshold 266B.

In the determination of payment increase action 7320, the analysis unit 220 transmits the payment increase action 7320 to the communication module 230 in step 7300-12 and outputs the payment increase action 7320 to the insurer computing system 5000 in step 7300-13. The insurer computing system 5000 receives the payment increase action 7320 at the processor 5100 and, in step 7300-7 as for the payment decrease action 7310, the processor 5100 updates the plan data 5310 particular to the patient 3000 in the plan database 5300 with the payment increase action 7320. The payment increase action 7320 may be an increase in an insurance premium and/or an increase in copayment or some other form of deterrent.

The payment decrease action 7310 and the payment increase action 7320 associate the cost of a health risk of a patient 3000 with the health index 136 of the patient 3000. The health index 136 can be similarly used by insurers 500 or entities other than insurers 500, such as a health plan manager of a health system, to associate other determinations or projections of risk and cost, such as those for hospitalizations, for a particular patient 3000 or patient population 300 with the health index 136 of the patient 3000 or an aggregate of the health indices 136 of the patient population 300.

In the determination of a program recommendation 7330, the analysis unit 220 transmits the program recommendation 7330 to the communication module 230 in step 7300-14 and outputs the program recommendation 7330 to the patient device 3200 in step 7300-15. The patient device 3200 receives the program recommendation 7330 at the processor 3210 and, in step 7300-16, the processor 3210 may display the program recommendation 7330 on the display interface 3230, set a reminder or plurality of reminders for the program recommendation 7330 in the reminders 3240 application, and/or schedule the program of the program recommendation 7330 in the calendar 3250 application. In an exemplary embodiment, the program recommendation 7330 may be a weight loss program from the insurer 500. In another embodiment, the program recommendation 7330 may be presented to the patient 3000 with a notification that scheduling and completion of the program recommendation 7330 would increase the patient's health index 136 by a predetermined number of points. Changes in health indices 136 may be tracked corresponding to each program recommendation 7330 and stored in the program database 5400 to monitor the effectiveness of the program contained in the program recommendation 7330 to promote health behavior changes.

The plurality of patient populations 300, the plurality of providers 400, and the plurality of insurers 500 described with respect to the shown embodiment are merely exemplary. In other embodiments, additional or alternative stakeholders such as care managers, health plan administrators, and quality assessment experts may similarly contribute to the accumulated health data 2000. Further, each additional or alternative stakeholder known to those with ordinary skill in the art may access the health index 136 and the data quality indicator 142 for each patient 3000 in each patient population 300 at the health information system 200 as similarly described above for the providers 400 and insurers 500, may display the health index 136 and associated information as similarly described above, and may execute the health actions 7000 as similarly described above. Some embodiments of the display interfaces for these stakeholders may focus on information of particular interest to that stakeholder; for example, a display interface for the quality assessment expert may show aggregate health indices 136 and changes in the aggregate health indices 136 over time for patient populations 300 organized by specific provider 400 or other entity such as a clinic or health system.

What is claimed is:

1. A healthcare resource, comprising:
a health information system corresponding to a plurality of providers, a plurality of insurers, and a patient population including a plurality of patients, the health information system having:
(a) a behavior database storing an accumulated health data for each patient of the patient population, the accumulated health data including data from the providers, the insurers, and the patients,
(b) a first index database storing a plurality of health indices with each health index corresponding to one patient of the patient population, and
(c) an analysis unit connected to the first index database and having a first processor determining a health action executed at at least one of the providers, the insurers, and the patients, the health action based on the health indices stored in the first index database; and
a health index system having a second processor which executes:
(a) a behavior unit configured to receive the accumulated health data from the behavior database and filter the accumulated health data into a patient modifiable data,
(b) an index unit configured to receive the patient modifiable data from the behavior unit and calculate the plurality of health indices from the patient modifiable data, and
(c) an output unit configured to receive the plurality of health indices from the index unit and transmit the plurality of health indices to the first index database of the health information system.

2. The healthcare resource system of claim 1, further comprising a plurality of health information systems each corresponding to a different plurality of providers, a different plurality of insurers, and a different patient population including a different plurality of patients, the behavior unit configured to receive an accumulated health data from each of the plurality of health information systems.

3. The healthcare resource system of claim 1, wherein the behavior unit is configured to filter the accumulated health data by including in the patient modifiable data only data related to clinical and non-clinical patient modifiable behaviors.

4. The healthcare resource system of claim 3, wherein the index unit is configured to calculate each health index from a plurality of subcomponents.

5. The healthcare resource system of claim 4, wherein each of the subcomponents has a weighted subcomponent score determined from a subcomponent score and a subcomponent weight, and the index unit sums the weighted subcomponent scores to calculate the health index.

6. The healthcare resource system of claim 5, wherein the index unit is configured to calculate the subcomponent score of each of the subcomponents from a plurality of behavior categories each having a weighted behavior category score.

7. The healthcare resource system of claim 6, wherein the index unit is configured to calculate each weighted behavior category score from a plurality of health behavior measures each having a health behavior measure score.

8. The healthcare resource system of claim 7, wherein the index unit is configured to determine the health behavior measure score from at least one data element in the patient modifiable data.

9. The healthcare resource system of claim 8, wherein the health index system has a data quality unit and the second processor executes the data quality unit configured to determine a data quality indicator from the patient modifiable data by comparing a plurality of first data elements in the patient modifiable data with a plurality of second data elements capable of being incorporated into at least one subcomponent score.

10. The healthcare resource system of claim 1, wherein the health index system has a second index database connected to the second processor and storing the health indices calculated over time for each patient.

11. The healthcare resource system of claim 10, wherein the health index system has a patient activation unit and the second processor executes the patient activation unit configured to determine a patient activation indicator based on a pattern of health behavior in the patient modifiable data of the health indices stored in the second index database over time for each patient.

12. The healthcare resource system of claim 8, wherein each patient has a patient device with a patient processor, a patient memory storing a patient interface, and a patient display interface.

13. The healthcare resource system of claim 12, wherein the first processor executes the analysis unit configured to retrieve the health index particular to one patient from the first index database and transmit the health index to the patient device of the one patient.

14. The healthcare resource system of claim 13, wherein the patient processor incorporates the health index particular to the one patient into the patient interface and executes the patient interface to display the patient interface on the patient display interface.

15. The healthcare resource system of claim 14, wherein the patient interface has the health index, the subcomponents, and the subcomponent scores displayed on the patient display interface.

16. The healthcare resource system of claim 15, wherein the patient interface has the behavior categories, the health behavior measures, and the at least one data element of the patient modifiable data displayed on the patient display device.

17. The healthcare resource system of claim 14, wherein the health information system has a threshold database storing a plurality of score thresholds, a plurality of appointment thresholds, and a plurality of insurer thresholds.

18. The healthcare resource system of claim 17, wherein the health action is a score alert and the first processor executes the analysis unit configured to retrieve the score thresholds from the threshold database, compare the score thresholds to the health index and/or subcomponent scores of the one patient, and determine the score alert for each instance of the health index and/or subcomponent scores exceeding the score threshold.

19. The healthcare resource system of claim 18, wherein the first processor executes the analysis unit configured to transmit the score alert to the patient device, the patient processor incorporating the score alert particular to the one patient into the patient interface.

20. The healthcare resource system of claim 17, wherein each provider has a provider computing system with a provider processor, a provider memory storing a provider interface, and a provider display interface.

21. The healthcare resource system of claim 20, wherein the first processor executes the analysis unit configured to retrieve the health index particular to one patient from the first index database and transmit the health index to the provider computing system of the provider particular to the one patient.

22. The healthcare resource system of claim 21, wherein the provider processor incorporates the health index particular to the one patient into the provider interface and executes the provider interface to display the provider interface on the provider display interface.

23. The healthcare resource system of claim 22, wherein the health action is an appointment alert and the first processor executes the analysis unit configured to retrieve the appointment thresholds from the threshold database, compare the appointment thresholds to a plurality of previous appointment dates of the one patient, and determine the appointment alert for each instance of the previous appointment dates exceeding the appointment threshold.

24. The healthcare resource system of claim 23, wherein the first processor executes the analysis unit configured to transmit the appointment alert to the provider computing device, the provider processor incorporating the appointment alert particular to the one patient into the provider interface.

25. The healthcare resource system of claim 17, wherein the health action is one of a plurality of insurer actions and the plurality of insurer thresholds include a plurality of positive insurer thresholds and a plurality of negative insurer thresholds.

26. The healthcare resource system of claim 25, wherein each insurer has an insurer computing system with an insurer processor, an insurer memory, a plan database storing a plan data on an insurance plan for each of the plurality of patients, and a program database storing a plurality of health-based incentive programs of the insurer.

27. The healthcare resource system of claim 26, wherein the first processor executes the analysis unit configured to receive the positive insurer thresholds from the threshold database, compare the positive insurer thresholds to the health index and/or subcomponent scores of one patient, and determine a payment decrease action for each instance of the health index and/or subcomponent scores exceeding the positive insurer threshold.

28. The healthcare resource system of claim 27, wherein the first processor executes the analysis unit configured to transmit the payment decrease action to the insurer computing system, the insurer processor incorporating the payment decrease action into the plan data of the one patient to decrease a monthly premium or a copayment of the one patient.

29. The healthcare resource system of claim 26, wherein the first processor executes the analysis unit configured to receive the negative insurer thresholds from the threshold database, compare the negative insurer thresholds to the health index and/or subcomponent scores of one patient, and determine a payment increase action and/or a program recommendation for each instance of the health index and/or subcomponent scores exceeding the negative insurer threshold.

30. The healthcare resource system of claim 28, wherein the first processor executes the analysis unit configured to transmit the payment increase action recommendation to the insurer computing system, the insurer processor incorporating the payment increase action into the plan data of the one patient to increase a monthly premium or a copayment of the one patient.

31. The healthcare resource system of claim 28, wherein the first processor executes the analysis unit configured to transmit the program recommendation to the patient device, the patient processor displaying the program recommendation at the patient display interface.

32. The healthcare resource system of claim 1, wherein the health information system further corresponds to a plurality of stakeholders, each stakeholder contributing to the accumulated health data and capable of receiving the health indices from the health information system.

33. A health index system, comprising:
a processor which executes:
a behavior unit configured to receive an accumulated health data for a patient from a behavior database of a health information system and filter the accumulated health data into a patient modifiable data by including in the patient modifiable data only data related to clinical and non-clinical patient modifiable behaviors;
an index unit configured to receive the patient modifiable data from the behavior unit and calculate a health index for the patient from the patient modifiable data; and
an output unit configured to receive the health index from the index unit and transmit the health index to an index database of the health information system.

34. The health index system of claim 33, wherein the accumulated health data includes data from the patient, a plurality of providers corresponding to the health information system, and a plurality of insurers corresponding to the health information system.

35. A method, comprising the steps of:
receiving an accumulated health data for a patient from a behavior database of a health information system;
filtering the accumulated health data into a patient modifiable data by including in the patient modifiable data only data related to clinical and non-clinical patient modifiable behaviors;
calculating a health index for the patient from the patient modifiable data; and
transmitting the health index to an index database of the health information system.
